(19) United States
(12) Patent Application Publication
(10) Pub. No.: US 2025/0049078 A1
(43) Pub. Date: Feb. 13, 2025

(54) ECO-FRIENDLY ADDITIVES FOR HAY AND SILAGE INOCULANTS MINIMIZING GREENHOUSE GAS EMISSIONS

(71) Applicant: Kansas State University Research Foundation, Manhattan, KS (US)

(72) Inventor: Jeongdae Im, Manhattan, KS (US)

(21) Appl. No.: 18/720,832

(22) PCT Filed: Dec. 22, 2022
(86) PCT No.: PCT/US2022/082198

§ 371 Date: Jun. 17, 2024

Related U.S. Application Data
(60) Provisional application No. 63/292,750, filed on Dec. 22, 2021.

(51) Int. Cl. A23K 30/18; C12N 1/20; C12R 1/01

(52) U.S. Cl. CPC A23K 30/18; C12N 1/205; C12R 2001/01

(57) ABSTRACT

The present invention relates to novel and eco-friendly additives for forage inoculants and to forage inoculants comprising the same. Notably, the forage inoculants, in addition to reducing non-CCh greenhouse gas emissions, are capable of fermenting forage while not reducing the nutritional value of the forage, thereby producing conserved forages with maintained nutritional values. Methods of reducing non-CCh greenhouse gas emissions produced during forage conservation are also described.

### CROSS-REFERENCE TO RELATED APPLICATIONS

The present application claims the priority benefit of U.S. Provisional Patent Application Ser. No. 63/292,750, filed Dec. 22, 2021, entitled ECO-FRIENDLY ADDITIVES FOR HAY AND SILAGE INOCULANTS MINIMIZING GREENHOUSE GAS EMISSIONS, incorporated by reference in its entirety herein.

### BACKGROUND

### Technical Field

The present disclosure relates to eco-friendly additives for forage inoculants and to forage inoculants comprising the same, which are notably capable of reducing non-CO₂ greenhouse gas emissions produced during the forage conservation process.

### Description of Related Art

Sustainable food production is a way of producing a continuous supply of safe and nutritious foods while minimizing its environmental impacts. Forages are plants or parts of plants eaten by herbivores, and sustainable forage management is critical to livestock industry. Forages can be harvested and conserved during the peak growing season for use in time of scarcity. Historically, and throughout the world today, forage conservation is a key element for productive and efficient livestock farming. There are two major practices in forage conservation: hay and silage. Hay is dried below 20% moisture and densely packed in a rectangular or round shape, inducing anaerobic conditions in the central region. Silage is another form of conserved forage and the main principle is anaerobic environment with high moisture content (50˜70%), which encourages fermentation. In short, conserved forages are nutrient-rich ecosystems that are maintained partly or completely under anaerobic conditions; hence, they can serve as ideal habitats for diverse microorganisms—especially those involved in the production of greenhouse gases. Particularly, forage conservation is the third-largest yet unaccounted nitrous oxide (N₂O) source in the agricultural sector. However, despite the high production volume and widespread commercial use, little is known about these ecosystems and their greenhouse gas emission potentials.

As a result, there is a need for forage inoculants that are capable of reducing non-CO₂ greenhouse gas emissions produced during the forage conservation process and do not reduce the nutritional value of the forage.

### SUMMARY OF THE INVENTION

The present disclosure is broadly concerned with an additive for a forage inoculant comprising at least two components capable of reducing non-CO₂ greenhouse gas emissions from forage conservation. In preferred embodiments, the at least two components are selected from the group consisting of a component capable of inhibiting microbial denitrification activity or methanogenesis, a component capable of facilitating the conversion of N₂O to N₂ by non-denitrifying N₂O-reducing organisms, an N₂O-reducing enrichment culture, and mixtures thereof.

In other embodiments, the present invention may be a forage inoculant, the forage inoculant comprising: starter bacteria capable of decreasing the pH in a quantity of forage to 5.5 or less; and an additive comprising one or more components capable of reducing non-CO₂ greenhouse gas emissions from forage conservation. In preferred embodiments, when applied to forage, the forage inoculant does not reduce the nutritional value of the forage.

In yet another embodiment, the present invention may be a method of reducing non-CO₂ greenhouse gas emissions from forage conservation, the method comprising: applying to a quantity of forage: (1) a starter bacteria capable of decreasing pH in the quantity of forage to 5.5 or less, and (2) an additive comprising one or more components capable of reducing non-CO₂ greenhouse gas emissions from forage conservation, wherein the additive has a concentration of about 0.05 to about 0.5 g per kg of forage.

### BRIEF DESCRIPTION OF THE DRAWINGS

FIG. 1 is a schematic drawing of the potential microbial processes in conserved forages responsible for N₂O emissions and the enzymes involved in each process.

FIG. 2 is a schematic drawing of the microbially driven CH₄ cycle and respective marker genes.

FIG. 3A is a graph illustrating N₂O production from corn, alfalfa, and sorghum silages.

FIG. 3B is a graph illustrating N₂O production from alfalfa silages using two different varieties (HVX MegaTron alfalfa from WINFIELD UNITED and HybriForce 3400 alfalfa from DAIRYLAND SEED) harvested in two different physiological stages (mid-bud and early flowering stages).

FIG. 3C is a graph illustrating CH₄ and N₂O production from forages under anerobic conditions.

FIG. 4A is a graph illustrating N₂O production from corn, alfalfa, and sorghum silages under different conditions.

FIG. 4B is a graph illustrating N₂O production from alfalfa silage amended with different concentrations of chlorate.

FIG. 4C is a graph illustrating total (cumulative) production volume of N₂O and CH₄ production simulated silage.

FIG. 5 is a schematic drawing of the genes associated with greenhouse gas cycles under aerobic conditions.

### DETAILED DESCRIPTION

The present invention concerns novel additives for forage inoculants and forage inoculants comprising the same. Also described are methods of reducing non-CO₂ greenhouse gas emissions produced during the forage conservation process.

### Additives

In one or more embodiments, the additives comprise, consist essentially of, or consist of one or more components capable of reducing non-CO₂ greenhouse gas emissions from forage conservation. As used herein, the term “non-CO₂ greenhouse gas” refers to any greenhouse gas (i.e., a gas that contributes to the greenhouse effect by absorbing infrared radiation) recognized and/or classified as such by the U.S. Environmental Protection Agency. Examples of non-CO₂ greenhouse gases include, but are not limited to, methane (CH₄), nitrous oxide (N₂O), and fluorinated gases (e.g., hydrofluorocarbons (HFCs), perfluorocarbons (PFCs), and sulfur hexafluoride (SF₆)). Preferably, the additives and methods of using the additives according to embodiments of the present invention are most concerned with reducing non-CO₂ greenhouse gases that are produced by a living organism.

In at least one embodiment, the one or more components capable of reducing non-CO₂ greenhouse gas emissions are capable of inhibiting microbial denitrification activity and/or methanogenesis. Examples of these components include, but are not limited to, analogs of nitrate reductase (e.g., chlorates and chlorites), phenylacetylene, and procyanidins. In preferred embodiments, the component capable of inhibiting microbial denitrification activity and/or methanogenesis is chlorate. In certain embodiments, the additive comprises about 1% to about 99% by weight, preferably about 20% to about 80% by weight, and more preferably about 40% to about 60% by weight of the component capable of inhibiting microbial denitrification activity and/or methanogenesis, wherein the % by weight is based upon the total weight of the additive taken as 100% by weight.

In at least one embodiment, the one or more components capable of reducing non-CO₂ greenhouse gas emissions includes a labile carbon source, which, if included, serves as an external carbon and/or energy source to alleviate low carbon-to-nitrogen ratios (i.e., one of the potential causes of N₂O accumulation during denitrification). In these embodiments, the component is preferably capable of facilitating the conversion of N₂O to N₂ by non-denitrifying N₂O-reducing organisms, such as denitrifiers, without N₂O accumulation. Examples of these components include, but are not limited to, labile carbon sources, carboxylic acids and their respective conjugate bases especially C₂-C₃ carboxylic acids including acetic acid (acetate) and lactic acid (lactate). In preferred embodiments, the component capable of facilitating the conversion of N₂O to N₂ by non-denitrifying N₂O-reducing organisms is acetate. In most embodiments, the additive comprises about 1% to about 99% by weight, preferably about 20% to about 80% by weight, and more preferably about 40% to about 60% by weight of the component capable of facilitating the conversion of N₂O to N₂ by non-denitrifying N₂O-reducing organisms, wherein the % by weight is based upon the total weight of the additive taken as 100% by weight.

In at least one embodiment, the one or more components capable of reducing non-CO₂ greenhouse gas emissions includes an N₂O-reducing enrichment culture, preferably inoculated with fermented silage and wastewater sludge at a pH of 5 or less. In embodiments where the one or more components include an N₂O-reducing enrichment culture, the culture is Gemmobacter serpentinus strain HB-1. In most embodiments, the additive comprises about 1% to about 20% by weight, preferably about 5% to about 15% by weight, and more preferably about 8% to about 10% by weight of the N₂O-reducing enrichment culture, wherein the % by weight is based upon the total weight of the additive taken as 100% by weight.

In some embodiments, the additive may comprise, consist essentially of, or consist of two or more components. In these embodiments, the first component is a component capable of inhibiting microbial denitrification activity and/or methanogenesis, preferably chlorate, and the second component is a component capable of facilitating the conversion of N₂O to N₂ by non-denitrifying N₂O-reducing organisms, preferably acetate. In some embodiments, the additive may further comprise a third component, which preferably is an N₂O-reducing enrichment culture.

In at least one of the foregoing embodiments, the additive may further comprise one or more inactive ingredients.

### Forage Inoculants

In one or more embodiments, the forage inoculants comprise, consist essentially of, or consist of the above-described additive(s), preferably the additive comprising chlorate or acetate, and starter bacteria capable of decreasing the pH in a quantity of forage to 5.5 or less. In preferred embodiments, the starter bacteria are members of the order Lactobacillales (i.e., lactic acid bacteria). Examples of starter bacteria include, but are not limited to, anaerobic lactic acid bacteria (e.g., Lactobacillus buchneri), Lactobacillus plantarum, Enterococcus faecium, and mixtures thereof. In most preferred embodiments, the starter bacteria are commercial inoculants containing lactic acid bacteria, such as Pioneer® 11H50, Pioneer® 1174, and Pioneer® 1129. In some embodiments, the forage inoculant comprises about 90% to about 99.99% by weight, preferably about 95% to about 99.9% by weight, and more preferably about 98% to about 99% of the additive and comprises about 0.01% to about 10% by weight, preferably about 0.1% to about 5%, and more preferably about 1% to about 2% by weight of the starter bacteria capable of decreasing the pH in a quantity of forage to 5.5 or less, wherein the % by weight is based upon the total weight of the forage inoculant taken as 100% by weight.

In at least one of the foregoing embodiments, the forage inoculant may further comprise one or more inactive ingredients.

The quantity of forage may be any plant material eaten by grazing livestock. In preferred embodiments, the quantity of forage is composed of highly digestible cell contents (e.g., protein, fat, soluble carbohydrates) and less digestible cell walls (e.g., cellulose, hemicellulose, lignin), which can be divided into two types: legumes and grasses. Legumes include, but are not limited to, Alfalfa (Medicago sativa), and grasses include, but are not limited to, corn (Zea mays) and sorghum (Sorghum bicolor). If the forage is ensiled, the silo/quantity of forage has a bulk density of about 400 kg/m³ to about 1000 kg/m³, preferably about 700 kg/m³.

The forage preservation/conservation process may be performed using any methods known in the art. In most embodiments, however, by lowering the pH of the quantity of forage to 5.5 or less, the forage inoculant ferments the quantity of forage, thereby forming conserved forage, which may be in the form of silage, haylage, or baleage. Particularly, in embodiments where in the starter bacteria are members of the order Lactobacillales, during the fermentation process, indigenous or exogenous lactic acid bacteria convert water-soluble carbohydrates to organic acids, mainly lactic acid, which act as a natural preservative to inhibit undesirable microorganisms. It will be appreciated that, when the forage inoculant is applied to the quantity of forage, the forage inoculant does not reduce the nutritional value of the forage.

### Method of Reducing Non-CO₂ Greenhouse Gas Emissions

In one or more embodiments, the method of reducing non-CO₂ greenhouse gas emissions comprises providing forage, preferably a quantity of forage, and applying the above-described forage inoculant. In these embodiments, the forage inoculant is applied to the quantity of forage at a loading of from about 0.001% by weight to about 0.2% by weight, preferably about 0.01% by weight to about 0.1% by weight, wherein the % by weight is based upon the total weight of the quantity of forage taken as 100% by wet weight. In preferred embodiments, the method of reducing non-CO₂ greenhouse gas emissions comprises providing a quantity of forage and applying the above-described starter bacteria to the quantity of forage before the above-described additive is applied. In these embodiments, the starter bacteria are applied to the quantity of forage at a loading of from about 0.000001% by weight to about 0.00005% by weight, preferably about 0.000001% by weight to about 0.00001% by weight, more preferably about 0.000001% by weight to about 0.000005% by weight, wherein the % by weight is based upon the total weight of the quantity of forage taken as 100% by wet weight. In these embodiments, the additive is applied to the quantity of forage at a loading of from about 0.015% by weight to about 0.15% by weight, preferably about 0.015% by weight to about 0.06% by weight, more preferably about 0.015% by weight to about 0.0275% by weight, wherein the % by weight is based upon the total weight of the quantity of forage taken as 100% by wet weight. The additive is applied about 1 minute to about 1 hour, preferably about 10 minutes to about 50 minutes, and more preferably about 20 minutes to about 40 minutes after the starter bacteria is applied to the quantity of forage.

Regardless of when the additive is applied, the concentration of the additive is about 0.015 to about 0.03 g per kg of forage, preferably about 0.015 g per kg of forage (wet weight based). In embodiments where the additive comprises two components, the first component is preferably chlorate at a concentration of about 0.05 g per kg of forage, and the second component is preferably acetate at a concentration of about 0.1 g per kg of forage. In most preferred embodiments, the method reducing of non-CO₂ greenhouse gas emissions further comprises incubating the forage after the forage inoculant and/or the starter bacteria and additive are applied.

Additional advantages of the various embodiments described herein will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present disclosure encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the disclosure. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### Examples

The following examples set forth methods in accordance with the disclosure. It is to be understood, however, that these examples are provided by way of illustration and nothing therein should be taken as a limitation upon the overall scope of the invention.

### Background

A preliminary study showed that a significant amount of N₂O, (38 mg CO₂ eq. per g-forage) and methane (CH₄, 0.2 mg CO₂ eq. per g-forage) were produced from laboratory incubations of alfalfa simulating silage process. Based on the annual silage production volume reported by USDA, the estimated N₂O emission potential was 0.3 million metric tons CO₂ equivalent per year, which places forage conservation as the third most important source of N₂O emissions in the agricultural sector. It should be noted that agriculture represents largest source of N₂O emissions comprising 79%. After 3-month of incubation, DNA was extracted and analyzed by polymerase chain reaction. All five genes involved in denitrification (narG, napA, nirK, norB, and nosZ) were detected, suggesting that denitrifying bacteria were responsible for N₂O emission and indicating that the conserved forages do harbor microbial communities involved in the production and reduction of GHGs.

This research promotes sustainable agriculture, which will not only address many environmental and social concerns, but also offer innovative and economically viable opportunities for growers, laborers, consumers, policymakers and many others in the entire food system. The forage silage inoculant market is estimated to be valued at $808M in 2019 and is projected to rapidly grow to $1.2B by 2025. Further, the findings of the proposed study may have tremendous economic consequences for international carbon credit trading, which involves assigning a monetary value to a reduction or offset of GHG emissions. The world bank reported that the carbon price should be set $100 per metric ton CO₂ eq by 2030. The estimated N₂O emission potential from forage conservation process—“the crisis” can be converted into “an opportunity” to earn extra revenues if efficiently controlled (0.3 million metric tons CO₂ eq per year×$100 per metric ton CO₂ eq=$30M per year). Based on the annual silage production volume in KS, the potential extra revenue is estimated to be $0.3M per year. Overall, this project will accelerate the transformation of livestock industry towards a healthy and sustainable global food system.

Justification. In most parts of the world, forage conservation is a key element for productive and efficient ruminant livestock farms. Forages provide the primary feed source for cattle, horses, and small ruminants. Forages are conserved to feed livestock during periods of shortage caused by limited pasture growth or inadequate pasture conditions. Historically, throughout the world, forage conservation is a crucial element for productive and efficient livestock farming. Forages are composed of highly digestible cell contents (protein, fat, soluble carbohydrates) and less digestible cell walls (cellulose, hemicellulose, lignin). The relative proportions of each component vary from crop to crop. Alfalfa (Medicago sativa), corn (Zea mays), and sorghum (Sorghum bicolor) are three major crops used for conservation purpose in the US.

Forage conservation methods can be primarily classified into hay and silage. Hay is dried below 20% moisture (12˜20%, w/w), baled in rectangular or round shape for ease of handling, transport, and storage, and stored under aerobic conditions. Silage is prepared with higher moisture content (40˜70%, w/w) and stored under strictly anaerobic conditions. Silage can be further subcategorized into silage, haylage, and baleage based on the moisture content. Silage is a high moisture forage containing 60-70% moisture. Haylage and baleage contain lower-moisture content (40-55%). Haylage is chopped using a silage chopper and packed in a bunk, silo, or bag. Haylage is called baleage when baled (before chopping) and wrapped with at least six layers of 1 mm plastic. Typically, alfalfa is conserved as both baled hay and silage, and corn and sorghum are only conserved as silage under anaerobic conditions. Forage preservation, especially silage, replies on fermentation. Indigenous or exogenous lactic acid bacteria (LAB) convert water-soluble carbohydrates to organic acids, mainly lactic acid, which act as a natural preservative to inhibit undesirable microorganisms. The optimal pH of silage is approximately 5.5 or below. The silage inoculant market is estimated to be valued at $808 million in 2019 and projected to rapidly grow to $1.2 billion by 2025.

Despite high production volume and widespread use, the forage conservation process has been scarcely studied as a possible source of GHGs. However, many researchers did detect CH₄ and N₂O. N₂O production was observed from corn silages stored in an open bunker silo, and the ambient N₂O concentration in the silo was orders of magnitude greater than for rural background levels. CH₄ and ammonia (NH₃) were detected in a dairy barn suggesting that the true culprit of the repeated fire incidents may be not the “elevated temperature” caused by microbial activity, but biogenic CH₄. A few other studies reported CH₄ and N₂O production from silage, but a comprehensive quantitative assessment has not been performed.

Forage Conservations. GHGs are the main contributor to the global warming and climate change due to their absorption of infrared radiation from the earth's surface. Although carbon dioxide (CO₂) constitutes the majority of GHG emissions, other non-CO₂ gases such as methane (CH₄) and nitrous oxide (N₂₀) also account for 25% of global GHGs emissions. N₂O is also a major source of ozone-depleting nitric oxide and nitrogen dioxide in the stratosphere and is currently the single most important ozone-depleting substance. Among others, agricultural activities are the largest anthropogenic source of N₂O comprising 79%. The emission will increase as it is closely linked to the most basic human need—food, unless substantial efforts are made to reduce the environmental footprint of agriculture.

Forages are plants or parts of plants eaten by grazing livestock, and they provide the main feed source for cattle, horses, and small ruminants. Forages can be conserved to feed livestock during periods of shortage caused by limited pasture growth or inadequate pasture conditions. Historically throughout the world, forage conservation is a key element for productive and efficient livestock farming. Typically, forages are conserved in the form of hay, usually below 20% moisture, and silage with high moisture content. Hay is packaged (or baled) with twine or plastic net in rectangular or round bales for ease of handling, transport, and storage. Rectangular bales are as small as 36 cm high, 46 cm wide, and 102 cm long, but they are usually stored as stacks in a covered storage facility. Typical round bales are larger, 1.2-1.8 m in diameter and 1.2-1.7 m in length. For both shapes, anaerobic regions are inevitably created in the core area. Silage is forage preserved by anaerobic storage, usually under conditions that encourage fermentation (1) to provide lactic acid as natural preservative to inhibit undesirable microorganisms by lowering pH, and (2) to improve nutritional value. Silages contain high moisture content (50-70%, w/w) and have several variations in preservation type including silage, haylage, and baleage primarily depending on their moisture levels. In short, conserved forages are nutrient-rich ecosystems that are maintained partly or completely under anaerobic conditions. Therefore, it was hypothesized that conserved forages can serve as ideal habitats for diverse microorganisms not only for fermenters but also those involved in the production of GHGs, e.g., methanogens and denitrifiers, that are known to produce CH₄ and N₂₀, respectively.

Microbial processes controlling N₂O. N₂O is the third-largest contributor of GHG emissions to the atmosphere, after CO₂ and CH₄. N₂O is also a major source of ozone-depleting nitric oxide (NO) and nitrogen dioxide (NO₂) in the stratosphere, N₂O emission currently is the single most important ozone-depleting emission. N₂O is produced by both natural and anthropogenic sources, but the emissions from human activities have increased 20% since the pre-industrial era, and its concentration has increased 15% since 1750. Approximately 40% of N₂O emissions come from human activities, and of those, the majority are from agricultural practices. In the US, about 75% of all anthropogenic N₂O emissions are attributed to agriculture.

Although the mechanism of N₂O production is not completely understood, microbial production of N₂O is generally considered to be achieved through three different processes: (1) a by-product of nitrification by ammonia-oxidizing bacteria, (2) denitrification, and (3) nitrifier denitrification, as well as possibly by (4) anaerobic ammonia oxidation and (5) nitrification by ammonia-oxidizing archaea (see FIG. 1). Among them, denitrification is a sequence of reductive reactions and is thought to occur mostly in anaerobic environments. For this reason, the potential for N₂O production from denitrification process is highest in anaerobic ecosystems.

Under anaerobic conditions, microbial denitrification is known to be the primary source of N₂O emissions. Denitrification is one of the key processes in nitrogen cycle, where nitrate (NO₃⁻) or nitrite (NO₂⁻) are reduced to molecular nitrogen (N₂) via the gaseous intermediates, NO and N₂O. Each reduction step is catalyzed by one or more specific reductase enzymes encoded by narG, napA, nirK, nirS, norB, and nosZ genes. The accumulation of denitrification intermediates and the subsequent emission of N₂O to the atmosphere are highly undesirable. Although the mechanism of this phenomenon still needs to be clarified, several factors have been reported to result in N₂O accumulation during denitrification, including low pH, low C/N ratios, the presence of elevated levels of free nitrous acid or H₂S, and low oxygen concentrations. On the consumer side, the only known sink for N₂O in the biosphere was its enzymatic reduction to N₂ by N₂O reductase, and this reaction has been attributed exclusively to denitrifying microorganisms. However, recent reports have demonstrated that diverse microbial taxa possessing divergent nos clusters with genes that are related yet evolutionarily distinct from the typical nos genes of denitirifers. Their environmental role in controlling N₂O emissions remains undefined, and these non-denitrifying N₂O-reducers may play an important role under conditions that are not favorable (e.g., low pH) for denitrifiers.

It should also be noted that different N-compounds are known to inhibit methanogenesis, which catalyze the terminal step of organic matter degradation to CO₂ and CH₄. Any change in denitrification activity may induce higher methanogenic activity.

Non-denitrifying N₂O reducers. On the consumer side, there is only one known sink for N₂O in the biosphere—the microbial reduction of N₂O to N₂, catalyzed by the N₂O reductase (N₂OR). This enzyme has typically been found among bacteria and archaea performing complete denitrification (microbes possessing “clade I” nosZ, a gene encoding N₂OR). However, recent reports have demonstrated that diverse microbial taxa possessing divergent nos clusters with genes that are related yet evolutionarily distinct from the typical nos genes of denitirifers. Their environmental role in controlling N₂O emissions remains undefined, and these non-denitrifying N₂O-reducers may play an essential role under conditions that are not favorable for denitrifiers, such as, low pH.

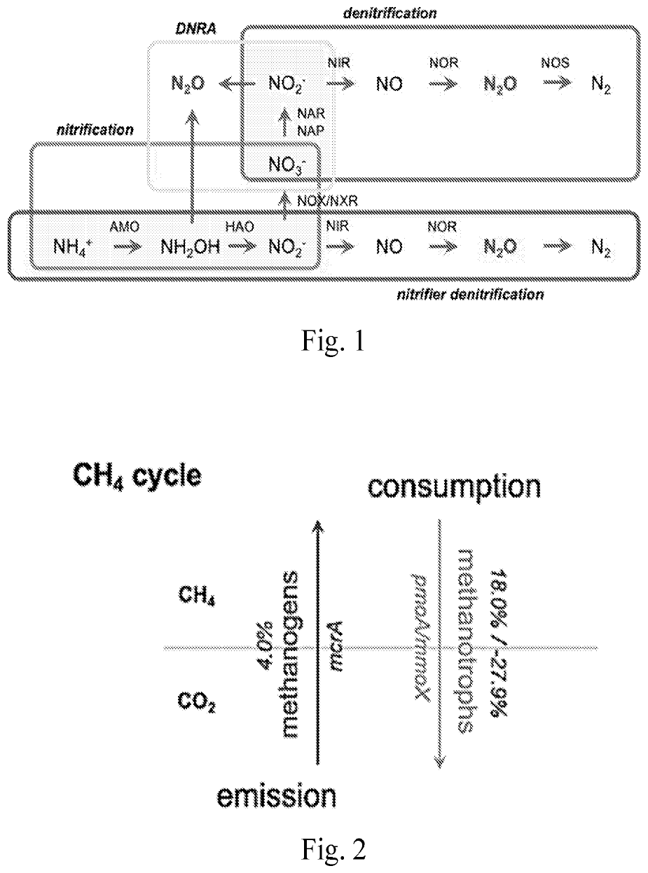

Microbial processes controlling CH₄. CH₄ is the second most important anthropogenic GHG after CO₂, contributing about 30% to the total net anthropogenic radiative forcing. The atmospheric concentration of CH₄ has been increasing from pre-industrial values of about 700 ppbv to currently about 1843 ppbv. Approximately 70% of the global CH₄ budget, which is on the order of 500-600 Tg CH₄ per year, is mainly the result of environmental microbial processes, such as methanogenesis in anaerobic environments and microbial CH₄ oxidation (methanotrophy) under anoxic and oxic conditions (see FIG. 2). Methanogenesis is anaerobic respiration that generates CH₄ as the final product of metabolism, and microbes capable of producing CH₄ are comprised exclusively of archaea, although many of them are closely associated with bacteria, e.g., fermentative bacteria. Methanogens can be subdivided into two primary functional groups: hydrogenotrophic methanogens (which use H₂ and CO₂ to produce CH₄) and acetoclastic methanogens (which use acetate to produce CH₄). All known methanogens express an isozyme of methyl-coenzyme M reductase (MRT); the mcrA gene encoding the α-subunit of this enzyme is commonly used as a “functional gene” marker for the detection, enumeration, and classification of methanogens (see FIG. 2).

On the consumer side, CH₄ can be oxidized by methanotrophs, a group of bacteria that utilize CH₄ as its sole carbon and energy source. Although acidophilic bacteria belonging to the phylum Verrucomicrobia was recently isolated, traditionally, methanotrophs are classified into two classes gammaproteobacterial (or Type I) and alphaproteobacterial (or Type II) based on several characteristics such as cell morphology, membrane arrangement, carbon assimilation pathway, and predominant phospholipid fatty acids (PLFA). Despite the diversity of methanotrophs and the wide range of environments in which they are found, the general pathway by which these cells oxidize CH₄ to CO₂ is remarkably similar with methanol, formaldehyde, and formate as intermediates. The first step of the reaction, CH₄ oxidation, is catalyzed by methane monooxygenase (MMO). The MMO can be found in two different forms, particulate (pMMO) and soluble (sMMO) forms. The a subunit of pMMO, encoded by the pmoA gene, is present in all known methanotrophs except for the genus Methylocella and Methyloferula spp., and is commonly used as a genetic marker for methanotrophs (see FIG. 2). Methanotrophs represent a major biological sink for CH₄ and are thus Earth's natural protection against this potent greenhouse gas.

Fire and explosion risks of CH₄ emissions. Repeated fire incidents have been reported from conserved forages, and “elevated temperature” caused by microbial activity has been generally considered as a culprit. The temperatures as high as 75° C. has been reported before bacterial activity ceases. It should be, however, noted that CH₄ is colorless, odorless, yet flammable gas with a lower explosive limit (LEL) of 5%. If ignited, CH₄ can pose a fire or explosion risk to people, infrastructure, or vegetation located nearby. The CH₄ flux measurement may decipher the causes of unexplained fire incidents at forage storage sites.

Factors affecting GHG emissions from conserved forages. GHGs production and consumption in conserved forages are controlled by microbial activity, and there are a variety of environmental factors affecting microbial growth including moisture content and nutrient availability.

In forage conservation practices, moisture content is governed by forage conservation method and nutrient availability varies from species to species. In the preliminary study, it was demonstrated that different cultivars and maturity stages do not affect the GHG production, and different crops and moisture content (i.e., conservation method) are used as independent variables.

### Preliminary Studies

In preliminary studies, the greenhouse gas (GHG) emissions were measured from simulated silage and baled hay. The effects of different treatments were evaluated on GHG emissions, and microbial communities were examined using quantitative polymerase chain reaction (qPCR) and functional gene microarrays.

GHG production from simulated silage. Laboratory experiments were performed to estimate the GHG emission potentials from silage conservation (i.e. anaerobic conservation). In a first part of the study, N₂O production from forage silos was studied. The forages used were corn, alfalfa, and sorghum. The mini silos consisted of 1 L glass jars connected to a 1-L Tedlar bag. The forage was chopped into 2-3 cm pieces with a laboratory chopper and ensiled in laboratory mini silos at a bulk density of 700 kg/m³ (700 grams per silo). The experiments were performed in an incubator at 30° C. in the dark, and the produced gas volume and composition were measured periodically for 30 days.

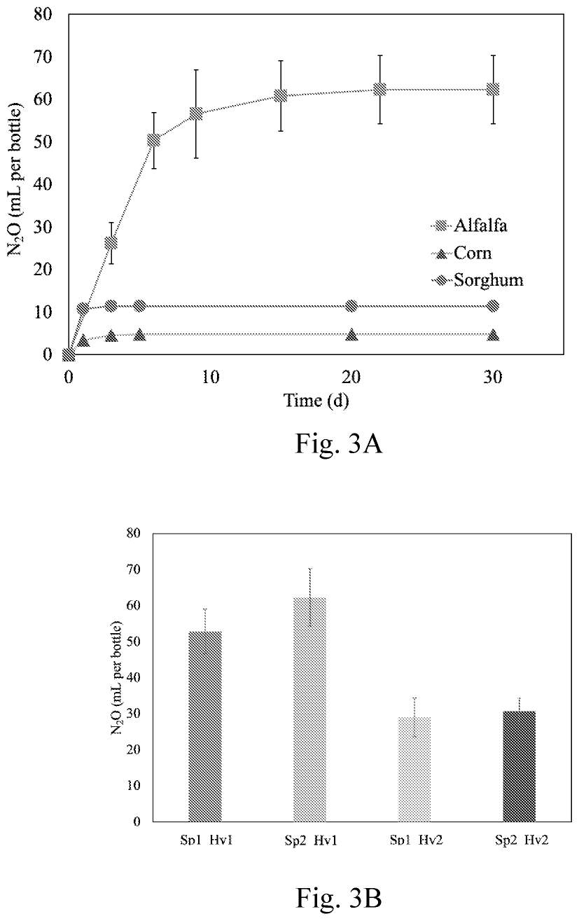

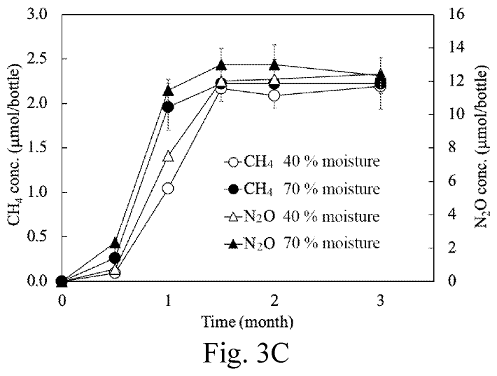

Three replicates for each crop were performed. Within a week of incubation, 4.1, 59.5, and 10.8 ml of N₂O were produced per bottle (see FIG. 3A). Two different alfalfa varieties including HVX MegaTron (WinField United LLC, Arden Hills, MN, USA) and HybriForce 3400 (Dairyland Seed Co.) were planted and harvested at two different maturity stages (mid-bud and early-flowering). There was no significant difference in N₂O production volume between the different varieties harvested at the same growth stages. However, the samples harvested at the later growth stages (i.e., early flowering) exhibited much lower N₂O production (see FIG. 3B).

In a second part of the study, N₂O and CH₄ production from forage silos were studied. The forage used was second cut alfalfa (HVX MegaTron) harvested at the early flower stage. The mini silos consisted of 1 L glass jars connected to a 1-L Tedlar bag. The forage was chopped into 2-3 cm pieces with a laboratory chopper and ensiled in laboratory mini silos at a bulk density of 700 kg/m³ (700 grams per silo). The experiments were performed in an incubator at 30° C. in the dark, and the produced gas volume and composition were measured periodically for 35 days. 49±1 ml N₂O and 0.019±0.001 ml CH₄ were produced within two weeks of incubation, corresponding to 113 mg and 1.4 μg CO₂ eq. per g-forage based on global warming potential of 300 and 25, respectively.

In a third part of the study, N₂O and CH₄ production from forage samples were studied. In 160 ml serum bottles, 20 g of forage samples were added, and the moisture contents were adjusted to 40% and 70% (w/w) by adding sterilized deionized distilled water. Each bottle was closed with black rubber stopper and capped with aluminum cap. All the bottles were incubated at 30° C. in the dark, and the headspace samples were monitored for CH₄ and N₂O for 3 months. Within 2 months of incubation, up to 2.2 (±0.3) μmol of CH₄ and 13.0 (±1.2) μmol of N₂O were produced, which corresponds to 122.2 and 8,581.2 μg CO₂ eq per g_{-forage}, respectively (see FIG. 3C). (In FIG. 3C, the error bars represent standard errors of triplicate experiments and are not visible when smaller than the symbols.) The production rates of both gases were slower with lower moisture content (40%) but the total concentrations at the end were similar regardless of the moisture content.

After the incubation (in parts 1 and 3 of the study), DNA was extracted using the method optimized for forage samples in the lab. PCR amplification was performed using primers targeting genes associated with denitrification (narG, napA, nirK, nirS, norB, and nosZ), and they were all positive.

Effects of different chemical treatments on GHG production. The treatments compared in this research consisted of (1) no treatment, (2) commercial silage inoculant (Pioneer® inoculant), (3) inoculant+chlorate (0.1%, w/w), and (4) inoculant+acetate (0.1%, w/w). Three replicates for each treatment were performed.

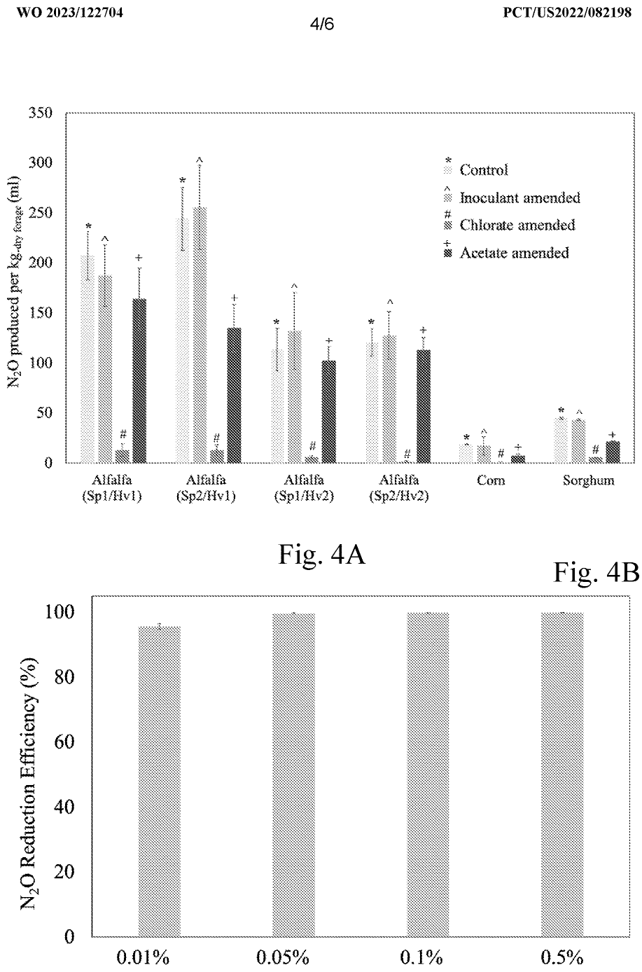

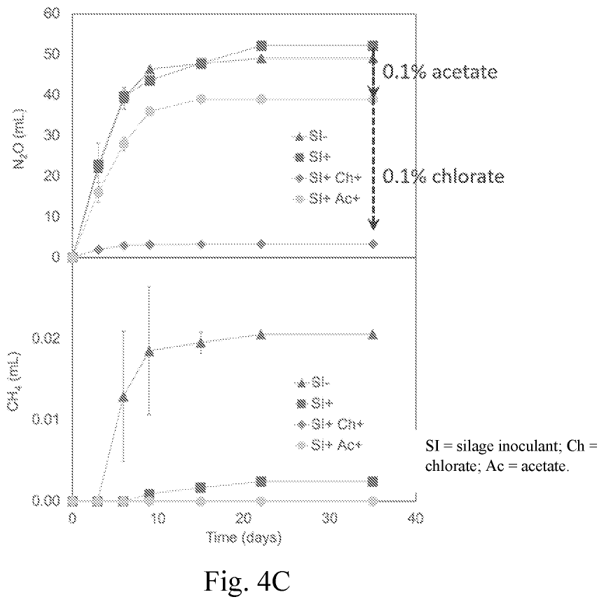

In the fourth part of the study, three replicates for each treatment were added to forage silos prepared as described in paragraph [0052]. As shown in FIG. 4A, the addition of the silage inoculant did not affect N₂O production. The addition of 0.1% (w/w) chlorate significantly reduced N₂O production by up to 99%, confirming that denitrifiers were responsible for N₂O production in this ecosystem. Acetate amendment also diminished N₂O production by up to 25% suggesting low C/N ratio could be one of the causes for N₂O accumulation. These results provide (1) insights into N₂O production mechanisms in the forage conservation process and (2) a basis to develop improved forage management practices minimizing environmental impact. FIG. 4B shows the effect of different concentrations of chlorate. Lower concentrations up to 0.01% (w/w) exhibited higher than 90% reduction of N₂O.

In the fifth part of the study, three replicates for each treatment were added to forage silos prepared as described in paragraph [0054]. In the control treatment (SI-), 49±1 ml N₂O and 0.019±0.001 ml CH₄ were produced within two weeks of incubation, corresponding to 113 mg and 1.4 μg CO₂ eq. per g-forage based on global warming potential of 300 and 25, respectively. As shown in FIG. 4C, the addition of the silage inoculant did not affect N₂O production but CH₄ production was significantly decreased, suggesting that low pH induced by the silage inoculant inhibited methanogenesis. The addition of 0.1% (w/w) chlorate significantly reduced N2O production by more than ten times, confirming that denitrifiers were responsible for N2O production in this ecosystem. Like in the fourth part of the study, the acetate amendment also significantly diminished N₂O production by 25% (P<0.01) suggesting low C/N ratio could be one of the causes for N₂O accumulation. Again, these results provide (1) insights into N2O production mechanisms in the forage conservation process and (2) a basis to develop improved forage management practices minimizing environmental impact.

GHG production from round hay bales. In the field experiments, two round alfalfa hay bales were monitored for surface GHG fluxes to evaluate the GHG emission potentials from hay. The surface fluxes were measured using a flow-through chamber method, which is the most common technique to measure surface GHG fluxes.

Briefly, a custom-built closed chamber with two gas ports was installed on the surface of round hay bales, and CH₄-free argon controlled by a flowmeter passed into the chamber at 5 mL/min. The chamber headspace was continuously mixed with a battery-powered fan installed inside. The outflow gas samples were collected, measured in the laboratory. The flux was calculated using the following equation, F=C·q/A, where F is the emission flux (μg/m²×hour), C is the concentration (μmol/m³), q is the air flow rate (m³/hour), and A is surface area (m²). CH₄ was not observed throughout the study, but N₂O was observed after two months of incubation. The concentrations were 0.26 and 0.13 μmol/L at 1 and 2 months of age, which correspond to 0.66 and 0.33 μmol/day/m², respectively. During each field sampling event, forage core samples were collected for molecular biological analysis using a hay coring probe at two different depths representing oxic region (5-25 cm) and anoxic/anaerobic region (35-55 cm), respectively, at 0, 1, and 2 months. After sampling, the holes were sealed with spray polyurethane foam to prevent an oxygen intrusion.

Given that CH₄ was not detected from the surface flux samples, it was hypothesized that CH4 was microbially oxidized by methanotrophs in the outer layer where oxygen (O₂) is available. To verify the hypothesis, 20 g of forage samples obtained from the outer layer of each bale were incubated with 10 μL of CH4 in 160 ml serum bottles. CH₄ in each bottle was completely disappeared in two days, indicating that methanotrophs are present in the outer layer.

Optimization of microbial nucleic acid extraction from forage samples. Analysis of phyllosphere microbial communities can be challenging due to the interference with plant DNA, especially when applying shotgun sequencing. To minimize the release of plant DNA during the DNA extraction, the microbial DNA extraction method was optimized using a proteinase K-based DNA extraction kit by varying the incubation time and temperature with the enzyme.

The copy number of 16S rRNA gene per ng DNA was used as a dependent variable. Briefly, 5 g of forage sample was mixed with 35 mL of TE-buffer (pH 8) and incubated on a shaker at 120 rpm for 15 min at room temperature. The mixture was centrifuged at 14,100 g at 4° C. for 20 min, and the resulting pellets were used to extract DNA using the Qiagen Blood and Tissue kit. After mixing with proteinase K, each sample was incubated at 40° C. for 20 min.

Microbial community analysis. DNA was extracted using the Blood and Tissue kit (Qiagen, Hilden, Germany), and Geochip 5.0S, a functional gene microarray, was used to examine the relative abundance of functional genes. Compared to the initial time point, genes associated with N cycles, i.e., amoA, hao, norB, napA, and nrfA, significantly increased in the anoxic/anaerobic region in a month, which coincided with the N₂O emissions.

After two months of incubation, presumably due to the depletion of nutrients, the N-related gene abundances plateaued or decreased (amoA, hao, and napA) (data not shown). CH₄ oxidation functional gene (pmoA) abundance increased in the oxic region of forage at one month, suggesting CH₄ was oxidized by methanotrophs and evaded detection on the surface. In conclusion, conserved forages do harbor microorganisms associated with N₂O and CH₄ cycles, and further research is warranted to comprehensively evaluate their role in controlling GHG emissions from the forage conservation process.

The forage materials contain organic compounds that spanned a wide range of recalcitrance from the labile starch to the recalcitrant lignin, allowing us to examine how active microbial communities utilized substrates with different recalcitrance. Within one month of incubation, the gene abundances associated with labile C degradation increased. After another month, the gene abundances associated with labile C degradation decreased and those associated with recalcitrant C degradation increased, presumably due to the depletion of labile organic C.

### Isolation of Novel N₂O-respiring Bacterium

With the goal to isolate microorganisms that can be used to minimize N₂O emissions from conserved forages, a novel N₂O-respiring bacterium was isolated from the round hay bales and characterized. A cream-colored colony found on the plate after four days of incubation, designated strain HB-1^{T}, was isolated using anoxic mineral salt medium buffered with 5 mM phosphate at pH 7 amended with 5 mM acetate and 5 mL of N2O as a sole electron acceptor. Strain HB-1^{T} had a unique cellular fatty acid composition with a substantially higher abundance of palmitic acid (C16:0). The average nucleotide identity values suggest that strain HB-1^{T} represents a new species. Further isolation efforts targeted at N2O reducing bacteria at low pH are in progress.

### Quantification and Mechanisms of Non-CO₂ Greenhouse Gas Emissions from Forage Conservation

As explained below, a temperature-controlled silage reactor is used to simulate the forage conservation process under anaerobic conditions. In the preliminary experiments, a significant volume of CO₂ was produced, which may disturb the microbial community unless properly released. A gas bag is connected to the headspace to collect the evolved gas and maintain atmospheric pressure in the reactor. The GHG emission potentials of conserved forages are assessed using three different crops; alfalfa, corn, and sorghum. These crops were selected because (1) alfalfa is legume and corn and sorghum are grass, and (2) they have different harvest windows, spaced at least two months apart. The moisture content is adjusted to 70%.

Chemical and biological treatments are evaluated for their impact on GHG emissions and nutrient contents. For the chemical treatments, chlorate and acetate are used. Low C/N ratio is one of the potential causes for N₂O accumulation during denitrification, and acetate serves as an external carbon source. For the biological treatment, N₂O-reducing enrichment cultures inoculated with fermented silage and wastewater sludge at low pH are used as an inoculant to consume N₂O.

The microbial community composition and their functional potential are investigated using a combination of culture-independent and culture-dependent approaches. Microbial diversities are assessed through culture-independent community analysis based on high coverage, high-throughput sequencing. Direct quantification of gene abundances and their transcripts through quantitative PCR (qPCR) and reverse transcriptase quantitative PCR (RT-qPCR), respectively, are performed. Key here is the use of transcript:gene abundance ratios as a measure of physiological activity for different microbial communities. This ratio can serve a direct predictor of GHG flux. In addition to the primer sets targeting bacterial/archaeal 16S rRNA genes, a functional gene related to CH₄ production (mcrA) and N₂O cycle (narG, napA, nirK, nirS, norB, nosZ, and nrfA) are used.

Quantification of non-CO₂ GHG emissions from silages. To quantitatively assess GHG emissions from silage, the three major silage crops, including alfalfa, corn, and sorghum, are ensiled at different moisture contents in a 1-L mini silo connected to a 1-L Tedlar bag at a bulk density of 700 kg/m³ as described above. The experimental design and harvesting windows of each crop are summarized in Table 1.

TABLE 1
Experimental design and harvesting window for each species.

| | | Moisture | |
|---|---|---|---|
| Experimental | Conservation | Content | Harvesting Window |
| Design Species | Method | (%, w/w) | in Kansas |
| Alfalfa | Hay/silage | 20*/40/55/70 | May-August |
| Corn | Silage | 40/55/70 | September-November |
| Sorghum | Silage | 40/55/70 | September-November |

The gas bags are replaced with new ones to characterize the temporal variations in gas production volume and composition on days 1, 2, 3, 7, 14, and 21. Whenever the gas bags are replaced, the total gas production volumes is measured by the water displacement method. The forage samples are collected and stored at −80° C. for chemical and molecular biological analysis. A subset of samples are submerged in RNAlater® buffer for RNA analysis. The detailed description of molecular analysis is presented below. The forage sample collection is carried out in the anaerobic chamber (Coy Laboratory Products, Ann Arbor, MI) containing a nitrogen/hydrogen (97/3; v/v) atmosphere to prevent any oxygen intrusion. The sampling frequency was determined based on the preliminary study. The inoculants are provided by Pioneer Hi-Bred International, Inc. The alfalfa, corn, and sorghum samples are provided by Thurlow Farms Inc. Fresh harvest is wilted to targeted moisture content (45, 55, and 70%) and chopped into approximately 1.5-cm length particles.

Low concentration of CH₄ are measured using Agilent 7890 gas chromatograph equipped with a Flame Ionization Detector and a DB-624 capillary column (60 m×0.32 mm×1.8 m), and high concentration of CH₄ and CO₂ are measured using Shimadzu GC 2010 Plus equipped with Thermal Conductivity Detector (TCD). For N₂O measurement, the Agilent 7890 gas chromatograph system equipped with an electron capture detector and HP-PLOT/Q column (30 m×0.53 mm×40 μm) is used. For inorganic N (i.e., NH₄⁺, NO₃⁻ and NO₂⁻) and pH, “haylage juice” is prepared by mixing the forage sample and water (1:1, w/w). Dionex ICS 5000 ion chromatography is used for inorganic N measurement.

Quantification of non-CO₂ GHG emissions from hay. In the preliminary experiment, surface N₂O emission was monitored but accurate estimation was challenging due to the irregular shape of the surface. To better assess GHG emissions from hay (Table 1), an open circuit hay bale respiration chamber is installed at the K-State Beef Stocker Unit. The Unit has a metal-sided hay barn for indoor storage. A round bale (4 ft width×4 ft diameter) from second-cut alfalfa wrapped with plastic mesh wrap is placed inside of the respiration chamber. The chamber is built using bolted steel angles and flexible PVC sheets to form a gas-tight envelop. The inside dimensions of the chamber are 2 m (length)×2 m (width)×2 m (height). The chamber is continuously flushed with ambient air (20 ml·min⁻¹) to simulate the natural wind using Zero Air Generator HP2A-3500 (Parker-Balston, Haverhill, MA).

The chamber headspace is continuously mixed with a fan installed inside. The exhaust air escaping through the outlet on the opposite side is collected and measured in the laboratory. The flux is calculated using the following equation, F=(C·q), where F is the emission flux 30 (μg·hour⁻¹), C is the concentration (μg·m³), and q is the air flow rate (m³·hour). The ambient temperature and humidity of the headspace are monitored using a Govee H5072 Bluetooth thermometer (OH, USA).

Identification of mechanisms of non-CO₂ GHG emissions from silage. In the preliminary study, most N₂O was produced within the first week of silage incubation (FIG. 4), and the redox conditions of the silage were assumed to be microaerobic to anoxic. Under these conditions, both nitrification and denitrification can play roles in N₂O production.

Indeed, amoA (encoding ammonia monooxygenase subunit A) abundance also significantly increased in the anoxic region of the hay bales as well as denitrification gene abundances, suggesting that nitrifiers may play a role in N₂O emissions from silage. Several factors have been reported to result in N₂O accumulation during denitrification, including low pH, low carbon to nitrogen (C/N) ratios, consumption of internal storage compounds, the presence of elevated levels of free nitrous acid or H₂S, and low dissolved oxygen (DO) concentrations.

The types of carbon sources also affect N₂O accumulation. In this task, the cause of N₂O emission from silage process is investigated, focusing on pH, C/N ratio, and oxidation-reduction potential (ORP). A silage reactor is made using stainless steel cylinder (25 cmø×30 cm H). The reactor is made in-house at the Department of Electrical and Computer Engineering at K-State. Using a pH meter and ORP meter, pH and ORP, respectively, are recorded automatically on the computer through a data logger, and gas volume and composition are monitored manually using a water displacement gas collector. For the forage sampling, four sampling holes (1 cmø) are drilled on the sidewall of the reactor and closed using a rubber stopper. The samples are collected using a sample corer. Each sampling hole is sacrificed at each sampling event, and the holes are closed using the same rubber stopper. The samples are stored at −80° C. for molecular biological analysis, and a subset of samples is submerged in RNAlater® buffer for RNA analysis.

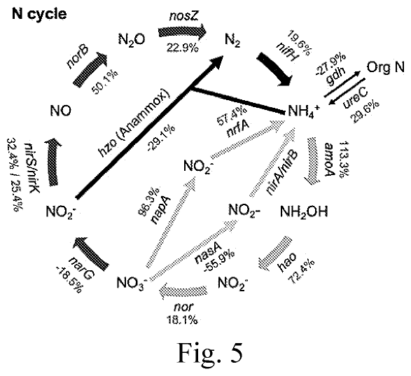

Characterization of the microbial community structure within conserved forages elucidates how they produce and reduce GHGs and eventually allow for estimates and control of GHG emissions. DNA are extracted from the samples collected (from the experiments performed as described above) for molecular analysis using the microbial nucleic acid extraction method optimized in the preliminary study as described above. To address the question of GHG fluxes in conserved forages are driven by changes in microbial communities, direct quantification of gene abundances and their transcripts through quantitative PCR (qPCR) and reverse transcriptase quantitative PCR (RT-qPCR) is performed, respectively. The key here is the use of transcript:gene abundance ratios as a measure of physiological activity for different microbial communities. This ratio can serve a direct predictor of GHG flux. In addition to the primer sets targeting bacterial/archaeal 16S rRNA genes, different functional genes of CH4 cycle—pmoA (encoding particulate CH4 monooxygenase), mmoX (soluble CH4 monooxygenase), and mcrA (α subunit of the methyl coenzyme M reductase), and N₂O cycle—narG (the membrane-bound nitrate reductase), napA (periplasmic nitrate reductase), nirK (copper-containing nitrite reductase), nirS (te cytochrome cd1 nitrite reductase), norB (nitric oxide reductase), nosZ (nitrous oxide reductase), nrfA (periplasmic nitrite reductase), and bacterial/archaeal amoA (ammonia monooxygenase) are used. For the samples obtained from the hay method, the genes associated with GHG cycles under aerobic conditions, i.e., bacterial/archaeal 16S rRNA, pmoA, mmoX, and bacterial/archaeal amoA, are examined in addition to mcrA, nrfA, and denitrification genes. All the primers except for pmoA, mmoX, and amoA are used for the silage samples. In addition to 16S rRNA, N cycle, and CH₄ cycle genes, genes associated with essential C-degradation (amyA, ara, pme etc.) are monitored as shown in FIG. 5.

A high-throughput metagenomic sequencing technique is used for in-depth community profiling. Reads are screened for accuracy using the Illumina quality control filter, and sequences shorter than 300 nt are removed from analysis to prevent inaccurate identification. Then, quality control of raw Illumina reads is performed, and sequences with imprecise matches to primer sequences, poor quality sequence reads, and sequences with ambiguous bases (≥1) are filtered out. Next, the Ribosomal Database Project II—FunGene (Functional Gene Pipeline and Repository) database is taken advantage of for accurate classification at the genus and family level for each gene. The database uses a reference sequence dataset and employs Hidden Markov models for alignment based on protein homology, allowing for statistical classification of query reads. Then, sequencing and analysis is performed as described above using both DNA and RNA (converted to cDNA) extracted from the samples. Analysis of DNA provides an estimation of the total microbial communities, while RNA analysis provides an estimate of that portion of the community physiologically active at the time of sampling. Both alpha (within-sample) and beta (among-samples) diversities are quantified to test hypotheses about variation in microbial communities along temporal and environmental gradients (crop type, moisture content). This is done using both taxonomic and phylogenetic methods. Taxonomic alpha diversity is measured as an operational taxonomic unit (OTU) and taxonomic group richness and equitability (Shannon diversity) within individual communities. Taxonomic dissimilarity of different communities is measured as the Bray-Curtis distance among samples based on OTU and taxonomic group membership. Phylogenetic diversity is measured based on a phylogenetic tree linking representative sequences from all OTUs. Phylogenetic relationships are inferred among representative sequences using FastTree software, which results in an approximate maximum likelihood tree. The resulting phylogeny is used to compute Faith's PD, the sum of branch lengths within samples. A standardized version of Faith's PD is computed based on comparing the observed PD to the PD expected under a random null model of community assembly to account for variation in the number of sequences in different samples. Phylogenetic beta diversity is measured using the UniFrac dissimilarity metric as implemented in QIIME.

To examine the effects of different treatments on forage nutritional quality, forage samples are sent to Rock River Laboratory, Inc., Watertown, WI, and are analyzed with NIR spectrophotometry and wet chemistry for dry matter, crude protein, crude fiber, total tract neutral detergent fiber digestibility, carbohydrates, and fermentation products. The same sample is sent to Hi-Bred International, Inc for pH and fatty acids analysis.

To find the strength of the relationship between the gas production and the quantified microbial populations (gene abundances) and between these populations and the chemical parameters, correlation analyses (with Pearson's correlation coefficient and Spearman's rank correlation coefficient) is performed. All quantitative data are examined by analysis of variance (ANOVA) and t-test among the control and treatment groups.

### Framework to Control of N₂O Emissions from Forage Conservations

In the preliminary study, the amendment of chlorate and acetate (0.1%, w/w) significantly repressed N₂O production (P<0.001). Chlorate has been known to inhibit microbial denitrification activity by blocking nitrate reductase. The same concentration of chlorate was used as a ruminant supplement to reduce E. coli O157 population, suggesting that chlorate is promising as a silage additive minimizing N₂O emissions. Also, a low C/N ratio is one of the potential causes of N₂O accumulation during denitrification. It is hypothesized that acetate served as an external carbon source to alleviate the possible low C/N ratio stress. Denitrifiers are known to produce N₂O at low pH. On the consumer side, recent reports have demonstrated that diverse microbial taxa possessing divergent nos clusters with genes that are related yet evolutionarily distinct from the typical nos genes of denitrifiers. Their environmental role in controlling N₂O emissions remains undefined, and these non-denitrifying N₂O-reducers may play an important role under conditions that are not favorable for denitrifiers (e.g., low pH). In this objective, it is investigated how GHG production can be reduced by regulating microbial processes.

Investigation of the Effects of Nitrification and Denitrification Inhibitors. Denitrification may contribute significantly to N₂O emissions in the silage process, and the contribution of nitrification is less likely. However, nitrification should not be ruled out, because forages are ensiled under aerobic conditions and low concentrations of residual oxygen may have favored a marginal metabolism of microaerobic bacteria. Further, in the preliminary study, most N₂O was produced within the first week where microaerobic condition is maintained (FIG. 3, Yang et al., in preparation). The same mini silos connected to a Tedlar bag (see Section “Quantification of non-CO₂ GHG emissions from silages” in Example 4) is used to evaluate the effect of different concentrations of chemical inhibitors that are known to inhibit nitrification and denitrification on GHG production. In this task, phenylacetylene and chlorate (as potassium salt) are used as inhibitors of nitrification and denitrification, respectively, and their effects on GHG emissions is examined. Phenylacetylene is added at a concentration of 0.01-1 mg per kg-forage (wet weight), and chlorate is added at a concentration of 0.01-1 g per kg-forage (wet weight). The gas volume and composition are periodically monitored, and the forage samples are collected for chemical and molecular analysis as described above. Chlorate and phenylacetylene concentrations are monitored using IC and GC-FID, respectively. In addition to the genes of interest listed in Example 4, cld gene (encoding chlorite dismutase) are monitored.

Investigation of the effects of carbon source amendment. Decrease in carbon sources increases N₂O emissions during denitrification as the various enzymes in that process compete for the electrons where nitrous oxide reductase is the weakest competitor, which ultimately leads to incomplete denitrification. A study using a pure culture of Alcaligenes faecalis verified that as soon as the availability of carbon compounds became exhausted and the culture entered starvation, the main product of denitrification was N₂O instead of N₂, and the conversion rate increased up to 64% of the total N-feed. In the preliminary study, the total amount of N₂O was reduced by 25% by the amendment of acetate (as a sodium salt, 1 g per kg-forage). In this task, acetate and lactate are evaluated as external carbon sources to reduce N₂O accumulation during denitrification at a concentration of 0.1-10 g per kg-forage (wet weight). The gas volume and composition are periodically monitored, and the forage samples are collected for chemical and molecular analysis, as described above.

Isolation of N₂O-reducing microorganisms at low pH. In general, the vast majority of microbial biodiversity cannot yet be maintained in laboratory culture. This is true on GHG-cycling microorganisms as well; only a small percentage of CH₄ cycling microbes are currently in culture collections, and non-denitrifier N₂O-reducing organisms were only recently discovered. Although multiple sources of N₂O exist, there is only one known sink for N₂O in the biosphere—the microbial reduction of N₂O to N₂, catalyzed by the N₂O reductase. This enzyme has traditionally been assigned to bacteria and archaea performing complete denitrification until recent discoveries unveiled the unexpectedly broad diversity of nosZ. nosZ genes of the novel clade II are often found in organisms lacking the genes encoding the key denitrification enzymes, namely nirK or nirS, indicating that these organisms utilize N₂O reduction as a respiratory reaction independent from denitrification. Such non-denitrifying N₂O reducers potentially can function as N₂O sinks. In the preliminary study, a novel N₂O-respiring bacterium, designated as Gemmobacter serpentinus strain HB-1, was isolated from a round hay bale.

For an application toward N₂O mitigation, characterization of more N₂O-reducing isolates is required. Efforts are continued on enrichment and isolation of N₂O-reducing bacteria and characterize their roles in conserved forages particularly at pH 5.5 or below (optimal silage pH). Further, the application of N₂O-reducing bacteria as a silage additive is examined. A continuous microbial enrichment is used as a tool to search for such unknown N₂O-reducing metabolisms. To develop enrichment cultures, 10 ml of sludge from a local wastewater treatment plant and 10 g of fermented silage samples is used to inoculate two double-jacket glass bioreactors (BioFlo®/CelliGen® 115, New Brunswick/Eppendorf, Germany) with 1 L working volume of sterile phosphate-buffered mineral salt media using N₂O as sole electron acceptor and acetate as sole carbon/energy source. Initially, organisms are grown under batch mode until biomass concentration reaches 1.2 g L⁻¹, and then continuous mode of operation is initiated. In one reactor, the initial pH is maintained at pH 7 and then gradually lowered to pH 5 by feeding fresh media adjusted to different pH. In another reactor, the pH is maintained at pH 5. The pH is monitored and maintained automatically by adding 1 M HCl and NaOH. The headspace N₂O concentration is monitored and maintained at a targeted level by feeding pure gas manually. Once enrichment cultures are obtained, purification is accomplished by repeated serial dilution with liquid media and streaking on solid media. At the same time, DNA is extracted and subjected to 16S rRNA gene amplicon sequencing. Both pure cultures and mixed enrichment cultures is used as an additive in simulated silage experiments (Task 1.1), and their effects on GHG emissions are examined.

### Validation of Laboratory Results by Performing Field Scale Evaluations

In order to validate the laboratory results, a field study is carried out at the K-State Beef Stocker Unit. The unit has 1,120 acres of native warm season grass subdivided into 20 functional paddocks. The unit has a well-drained site for outdoor forage storage and a metal-sided hay barn for indoor storage. The optimized forage amendment selected from the laboratory experiments is applied to prepare three silage bags (2.5 m×2.5 m) along with additional three bags as controls. Four gas bags are connected, apart from each other. Similar to the laboratory experiments, the gas bags are periodically replaced with new ones, and the gas composition and total volumes are monitored. The gas/forage samples are collected once in two weeks and used for chemical, nutritional, and molecular biological analyses.

The proposed studies in Examples 4 and 5 should provide insights into the GHG emission mechanisms from the forage conservation process and the strategies to control the emissions. In this objective, the results from these Examples are further validated in the field scale experiments at the K-State Beef Stocker Unit and the Thurlow Farms.

Round bale silages (baleages) made with alfalfa, corn, and sorghum are obtained and monitored for GHG production at the K-State Beef Stocker Unit. Baleage is a relatively new technique for conserving forage as silage, wrapped with at least 6 layers of 1 mil plastic and maintained under gas-tight conditions. Moisture content of the forage is adjusted to 50%. Two control baleages receive silage inoculant (Pioneer® 11H50, 1174, and 1129, respectively), and the other sets of two baleages are amended with treatments optimized in addition to the inoculant. Two gas bags (2 L capacity, SKC Inc. Eighty Four, PA) are connected on the other side. The forage samples are also collected for chemical and molecular analysis as described above. Similar to the laboratory experiments, the effect of different treatments on GHG emissions, silage nutritional quality, and microbial community are examined.

There are four bunker silos (20′W×8′H×80′L) for corn silage at the Thurlow Farms, and two of the silos are used for the study. The control silo receives silage inoculant (Pioneer® 1174), and the other silo is amended with treatments optimized in addition to the inoculant. The GHG emissions are monitored once a week by measuring the surface fluxes using a flow-through chamber method. The forage samples are collected for chemical and molecular analysis at each sampling event as described above. The effect of the treatment on GHG emissions, silage nutritional quality, and microbial community are examined.

1. An additive for a forage inoculant, the additive comprising at least two components capable of reducing non-CO₂ greenhouse gas emissions from forage conservation.

2. The additive of claim 1, wherein the at least two components are selected from the group consisting of a component capable of inhibiting microbial denitrification activity or methanogenesis, a component capable of facilitating the conversion of N₂O to N₂ by non-denitrifying N₂O-reducing organisms, an N₂O-reducing enrichment culture, and mixtures thereof.

3. The additive of claim 1, wherein the first component is a component capable of inhibiting microbial denitrification activity or methanogenesis and the second component is a component capable of facilitating the conversion of N₂O to N₂ by non-denitrifying N₂O-reducing organisms.

4. The additive of claim 2, wherein the component capable of inhibiting microbial denitrification activity or methanogenesis is chlorate or chlorite, and wherein the component capable of facilitating the conversion of N₂O to N₂ by non-denitrifying N₂O-reducing organisms is acetate or lactate.

5. (canceled)

6. The additive of claim 2, wherein the N₂O-reducing enrichment culture is Gemmobacter serpentinus strain HB-1 inoculated with fermented silage and wastewater sludge at a pH of 5 or less.

7. A forage inoculant, the forage inoculant comprising:
starter bacteria capable of decreasing the pH in a quantity of forage to 5.5 or less; and
an additive comprising one or more components capable of reducing non-CO₂ greenhouse gas emissions from forage conservation.

8. The forage inoculant of claim 7, wherein the starter bacteria are members of the order Lactobacillales.

9. The forage inoculant of claim 8, wherein the starter bacteria are selected from the group consisting of Lactobacillus buchneri, Pioneer® 11H50, Pioneer® 1174, and Pioneer® 1129, and mixtures thereof.

10. The forage inoculant of claim 7, when applied to the quantity of forage, the forage inoculant does not reduce the nutritional value of the quantity of forage, wherein the forage is selected from the group consisting of legumes and grasses.

11. The forage inoculant of claim 7, wherein the forage inoculant is capable of fermenting the quantity of forage, thereby forming conserved forage, wherein the conserved forage is in the form of silage, haylage, or baleage.

12. (canceled)

13. (canceled)

14. A method of reducing non-CO₂ greenhouse gas emissions from forage conservation, the method comprising:
applying to a quantity of forage:
(i) a starter bacteria capable of decreasing pH in the quantity of forage to 5.5 or less, and
(ii) an additive comprising one or more components capable of reducing non-CO₂ greenhouse gas emissions from forage conservation,
wherein the additive has a concentration of about 0.15 to about 0.3 g per kg of forage.

15. The method of claim 14, wherein the additive comprises one or more components that are selected from the group consisting of a component capable of inhibiting microbial denitrification activity or methanogenesis, a component capable of facilitating the conversion of N₂O to N₂ by non-denitrifying N₂O-reducing organisms, an N₂O-reducing enrichment culture, and mixtures thereof.

16. The method of claim 14, wherein the starter bacteria are applied to the quantity of forage before the additive.

17. The method of claim 15, wherein the component capable of inhibiting microbial denitrification activity or methanogenesis is chlorate or chlorite, and wherein the component capable of facilitating the conversion of N₂O to N₂ by non-denitrifying N₂O-reducing organisms is acetate or lactate.

18. (canceled)

19. The method of claim 15, wherein the N₂O-reducing enrichment culture is Gemmobacter serpentinus strain HB-1 inoculated with fermented silage and wastewater sludge at a pH of 5 or less.

20. The method of claim 14, wherein the additive comprises two components, wherein the first component is chlorate and the second component is acetate, and wherein the first and second components each have a concentration of about 0.015 g per kg of forage.

21. (canceled)

22. The method of claim 14, wherein the starter bacteria are members of the order Lactobacillales.

23. The method of claim 22, wherein the starter bacteria are selected from the group consisting of Lactobacillus buchneri, Pioneer® 11H50, Pioneer® 1174, and Pioneer® 1129, and mixtures thereof.

24. The method of claim 14, wherein the method further comprises incubating the quantity of forage.

25. The method of claim 14, wherein the quantity of forage has a bulk density of about 400 kg/m³ to about 1000 kg/m³, and wherein the non-CO₂ greenhouse gas is N₂O, CH₄, or combinations thereof.

26. (canceled)
